(19) United States
(12) Patent Application Publication
(10) Pub. No.: US 2016/0162655 A1
(43) Pub. Date: Jun. 9, 2016

(54) SYSTEMS AND METHODS FOR GUARDING AGAINST SIDE EFFECTS

(71) Applicant: KONINKLIJKE PHILIPS N.V., EINDHOVEN (NL)

(72) Inventors: John Cronin, Bonita Springs, FL (US); Seth Melvin Cronin, Milton, VT (US)

(21) Appl. No.: 14/957,238

(22) Filed: Dec. 2, 2015

Related U.S. Application Data
(60) Provisional application No. 62/087,102, filed on Dec. 3, 2014.

(30) Foreign Application Priority Data
May 26, 2015 (EP) 15169243.1

(51) Int. Cl. G06F 19/00; A61B 5/00

(52) U.S. Cl. CPC G06F 19/3418; G06F 19/326; G06F 19/3456; A61B 5/4848; A61B 5/0022; A61B 5/74; A61B 5/6801

(57) ABSTRACT

The present invention provides computer-implemented methods and systems for monitoring and informing a user about known side effects of drugs (e.g., prescription, over-the-counter) using a wearable device, the method including: polling for sensor data from a wearable device; the sensor data indicating one or more vital signs measurement parameters of a user, wherein the sensor data is used for evaluating whether one or more known side effects has been detected; transmitting a request for information to various networks, the information request including identification of at least one drug the user is using; receiving the requested information from the various networks for monitoring and detecting one or more known side effects of the identified drug; storing the received information from the various networks into memory of the wearable device; evaluating, for each identified drug, the obtained sensor data with the stored information received from the various networks pertaining to that identified drug; determining whether one or more known side effects have been detected, wherein the determination is based on the evaluation of the sensor data with the stored information about the identified drug; executing one or more actions to notify the user and other third parties based on the detected one or more known side effects; and executing an algorithm to adjust the drug delivery regime responsive to the determination that one or more known side effects have been detected.

### CROSS-REFERENCE TO PRIOR APPLICATIONS

This application claims the benefit of U.S. Provisional Patent Application No. 62/087,102, filed on Dec. 3, 2014 and European Patent Application No. 15169243.1, filed on May 26, 2015. These applications are hereby incorporated by reference herein.

### BACKGROUND OF THE INVENTION

1. Field of Invention

The present invention generally relates to wearable technology. More specifically, the present invention relates to using wearable technology to guard against side effects of prescription medication.

2. Description of the Related Art

Wearable technology may include any type of mobile electronic device that can be worn on the body, attached to or embedded in clothes and accessories of an individual and currently exist in the consumer marketplace. Processors and sensors associated with the wearable technology can display, process or gather information. Such wearable technology has been used in a variety of areas, including monitoring health data of the user as well as other types of data and statistics. These types of devices may be readily available to the public and may be easily purchased by consumers. Examples of some wearable technology in the health arena include Fit Bit, Nike Fuel Band, and the Apple Watch.

People use prescription drugs or over-the-counter drugs to deal with a variety of illnesses and conditions. Many of these drugs, however, may also have known side effects. These side effects are generally provided on standardized labels to inform the user. Individuals can also perform research regarding the side effects of medication using online tools (e.g., Web MD).

### SUMMARY OF THE CLAIMED INVENTION

It would be advantageous to have a system and computer-implemented method for monitoring and detecting one or more known side effects of drugs using a wearable device.

To better address this concern, a first aspect of the present invention includes computer-implemented methods for monitoring and detecting one or more known side effects of drugs using a wearable device. The methods includes polling for sensor data from a wearable device; the sensor data indicating one or more vital signs measurement parameters of a user, wherein the sensor data is used for evaluating whether one or more known side effects has been detected; transmitting a request for information to various networks, the information request including identification of at least one drug the user is using; receiving the requested information from the various networks for monitoring and detecting one or more known side effects of the identified drug; storing the received information from the various networks into memory of the wearable device; evaluating, for each identified drug, the obtained sensor data with the stored information received from the various networks pertaining to that identified drug; determining whether one or more known side effects have been detected, wherein the determination is based on the evaluation of the sensor data with the stored information about the identified drug; executing one or more actions to notify the user and other third parties based on the detected one or more known side effects; and executing an algorithm to adjust the drug delivery regime responsive to the determination that one or more known side effects have been detected.

A further aspect of the invention provides a wearable device for monitoring and detecting one or more known side effects of drugs. The wearable device includes a plurality of sensors, memory, and a processor configured for: polling for sensor data; the sensor data monitoring one or more vital signs measurement parameters of a user, wherein the sensor data is used for evaluating whether one or more known side effects has been detected; transmitting a request for information to various networks, the information request including identification of at least one drug the user is using; receiving the requested information from the various networks for monitoring and detecting one or more known side effects of the identified drug; storing the received information from the various networks into memory; evaluating, for each identified drug, the obtained sensor data with the stored information received from the various networks pertaining to that identified drug; determining whether one or more one or more known side effects have been detected, wherein the determination is based on the evaluation of the sensor data with the stored information about the identified drug; executing one or more actions to notify the user and other third parties based on the detected one or more known side effects; and executing an algorithm to adjust the drug delivery regime responsive to the determination that one or more known side effects have been detected.

The present invention is directed towards systems and methods for monitoring known drug-based side effects using a wearable device. In particular, the present invention utilizes information pertaining to known side effects and informs a user when sensor data monitoring health parameters of the user (e.g., vital signs measurement parameters such as pulse, blood pressure, temperature) detects the one or more known side effects. In other embodiments, pharmaceutical companies may also be able to use the wearable sensors and/or wearable sensor data to ensure that their drugs operate properly (e.g., as labeled or as required by some drug adherence/compliance regulation).

Therefore, the need in the art for improved systems and methods of wearable devices to guard against side effects is achieved. Data, obtained from the wearable device, could also be used in order to provide preventative health actions for the user to follow in view of the known side effects in order to lessen the effects of the side effects. Furthermore, networks could be provided that include information regarding various prescription drugs with their respective known side effects. Individuals using wearable devices may be able to utilize the information from the networks to stay informed about the medication that they may be using.

### BRIEF DESCRIPTION OF THE DRAWINGS

FIG. 1 illustrates an exemplary system for monitoring known drug-based side effects using a wearable device according to an embodiment of the present invention.

FIG. 2 shows a grid illustrating exemplary prescription drugs and their associated known side effects that may be monitored according to an embodiment of the present invention.

FIG. 3 illustrates an exemplary side effects database that may be found in the various networks according to an embodiment of the present invention.

FIG. 4 shows an exemplary method for the side effects software module found on the various networks according to an embodiment of the present invention.

FIG. 5 shows an exemplary method for the side effects software module found on the user device according to an embodiment of the present invention.

FIG. 6 illustrates an exemplary computing device that may be utilized to implement the various features and processes described herein according to an embodiment of the present invention.

FIG. 7 illustrates an exemplary method for monitoring and detecting known side effects of drugs according to an embodiment of the present invention.

### DETAILED DESCRIPTION

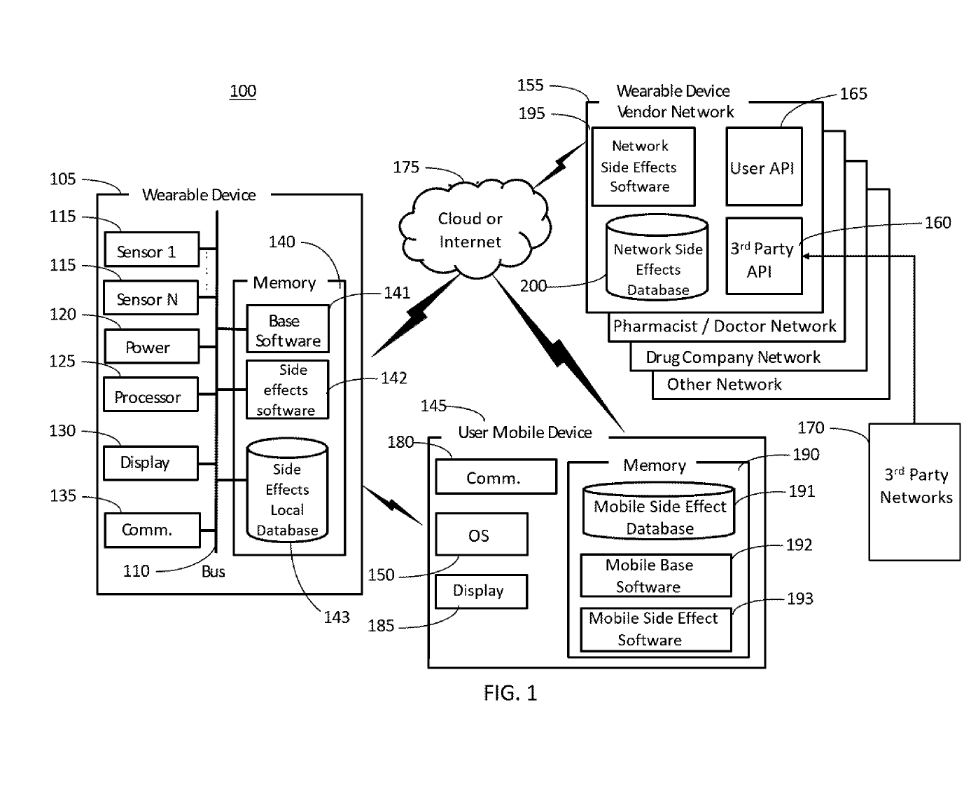

FIG. 1 illustrates an exemplary system for monitoring one or more known side effects using a wearable device. The system 100 may include a user wearable device 105, a user mobile device 145, and a plurality of networks 155. Each of the elements of the system 100 may be connected to the packet data network 175.

The wearable device 105 may be used by the user to obtain sensor data (e.g., vital signs measurement parameters) useful for detecting occurrences of known side effects of one or more drugs that the user may have used. The various known side effects may alter or influence changes in vital signs measurement parameters of individuals who use the drug. An example implementation of the present invention may include the user wearing the wearable device 105 after taking a particular drug. The wearable device 105 can then monitor various vital signs measurement parameters of the user based on possible known side effects the drug taken may have on the user. If the drug taken produces one or more known side effects, the wearable device 105 may be capable of detecting the known side effects from changes that occur in the vital signs measurement parameters of the user. The user can then be notified of the detected side effect.

The wearable device 105 may include a number of different elements all connected to a central bus 110. For example, the wearable device 105 may include a plurality of sensors 115, a power supply 120, a processor, 125, a display 130, communication module 135 and memory 140. It should be noted that the central bus 110 may facilitate communication between each of the elements of the wearable device 105. Even though FIG. 1 illustrates an embodiment where a single central bus 110 is used, there may be other embodiments where two or more bus connections are provided among each of the elements of the wearable device 105 to facilitate communication.

The wearable device 105 may include one or more sensors 115 mounted on the wearable device 105. The plurality of sensors 115 can be any type of sensor that is known in the art. Generally, sensors 115 can be used, for example, to detect and obtain sensor data (e.g., vital signs measurements) about the user (e.g., heart rate, blood pressure, temperature).

In an embodiment of the present disclosure, the sensors 115 may be used to monitor a health condition of the user based on sensor data of the vital signs measurement parameters (e.g., heart rate, blood pressure, temperature) of the user. A particular drug taken by the user may have one or more known side effects. Each of the side effects may have a known effect on one or more of the vital signs measurement parameters of the user. By using the sensor data, the wearable device 105 can detect occurrences of known side effects when they arise. In some embodiments, the sensor data may be used to detect possible occurrences of known side effects before they arise based, for example, on how the user vital signs measurement parameters are currently affected by the drug taken.

The wearable device 105 may include a power supply 120. The power supply 120 may be used to provide power used by the wearable device 105 for maintaining operation of the overall wearable device 105. An exemplary power supply 120 may include a battery. The wearable device 105 may also include a power supply 120 that can be charged/re-charged using an external power source (e.g., battery charger).

The wearable device 105 may also include a processor 125. In some embodiments, the wearable device 105 may include two or more processors. In any case, the processor 125 may be any computer processor known in the art. The processor 125 can be used to carry out the various instructions of the wearable device 105 (e.g., analysis of sensor data, calculations).

The display 130 included in the wearable device 105 may be used by the wearable device 105 to display various types of information or facilitate interaction between the user and the wearable device 105 (e.g., GUI). For example, in an embodiment, the user may use buttons or wheels (not shown) to select particular buttons or information displayed on the display 130 of the wearable device 105. In some embodiments, the display 130 may also be a touch screen display that may allow the user to directly interact with the wearable device through physical contact with the display 130.

The wearable device 105 may also include a communication module 135. The communication module 135 may facilitate communication (e.g., wireless communication) between the wearable device 105 and other devices (e.g., user mobile device) and/or networks 155. The communication module 135 may implement the communication through the use of one or more methods known in the art including Wi-Fi, Bluetooth, 3G, 4G, LTE, near field communication (NFC).

The memory 140 of the wearable device 105 may be used to store data associated with the wearable device 105. It should be noted that the memory 140 may also include various other software module and databases for carrying out the functionality of the wearable device 105. As illustrated in FIG. 1, the memory 140 may include base software module 141, side effects software module 142 and a side effects local database 143.

The base software module 141 of the wearable device 105 may be responsible for the management and operation of the wearable device 105. In an embodiment, the base software module 141 may poll for sensor data monitoring one or more vital signs measurement parameters of the user. The base software module 141 may also execute software module and other elements within the wearable device 105 to carry out the functionality of the wearable device 105. For example, the base software module 141 may instruct the wearable device 105 to obtain sensor data from one or more sensors 115. The sensor data from the sensors 115 can then be stored in the memory 140 to be later evaluated by the side effects software module 142.

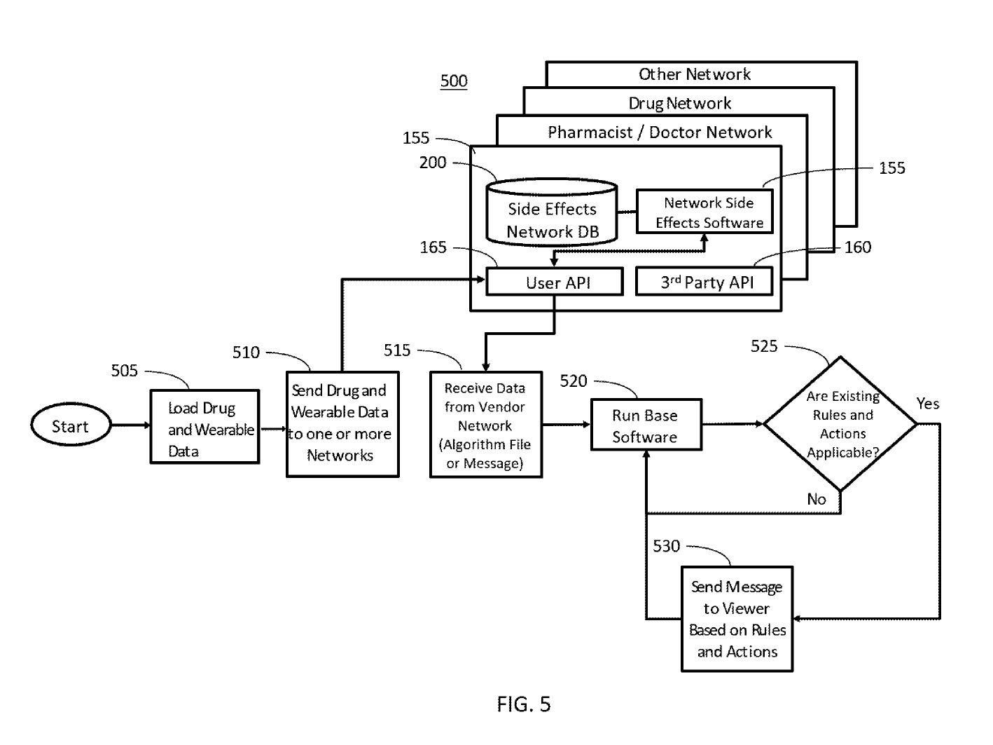

The side effects software module 142 may be used to process sensor data obtained by the sensors 115 of the wearable device 105 in conjunction with information stored in the side effects local database 143 to determine if one or more known side effects are present. The side effects software module 142 may also be used to update the side effects local database 143. In particular, the side effects software module 142 may update the side effects local database 143 by requesting the various networks 155 for related information that could be used based on the one or more drugs used by the user. Further details regarding the side effects software module 142 can be found, for example, in FIG. 5.

The side effects local database 143 may include information regarding known side effects of one or more various drugs that the user may be taking. The side effects local database 143 may include rules and/or algorithms that the wearable device 105 may use to evaluate the sensor data to detect the known side effects. As discussed in further detail below, the information stored in the side effects local database 143 may be downloaded from one or more networks 155 by connecting to the packet data network 175.

With respect to FIG. 1, the system 100 may also include the user mobile device 145. The user mobile device 145 may be used in conjunction with the wearable device 105 to assist in the various functions of detecting one or more known side effects of a drug used by the user. Exemplary user mobile devices 145 may include iPhone, Android or Windows type mobile devices. It should be noted that the user mobile device 145 may include elements that mirror the elements found in the wearable device 105. For example, the user mobile device 145 includes a communication module 180, a display 185, and memory 190. The memory 190 of the user mobile device 145 may also contain similar elements: the mobile side effect database 191, the mobile base software module 192, and the mobile side effect software module 193.

The elements illustrated in FIG. 1 that are common between the wearable device 105 and the user mobile device 145 may be provided so that both the wearable device 105 and the user mobile device 145 may be capable of performing, to varying extents, the same processes used for evaluating and detecting one or more known side effects. The user of the wearable device 105 and the user mobile device 145 may be allowed to customize user preferences dictating what aspects the present invention may be carried out using the wearable device 105 and what other aspects are carried out using the user mobile device 145. It may be possible, in an embodiment, that the wearable device 105 performs all the steps of the present invention without the use of the user mobile device 145. In other embodiments, for example, the user mobile device 145 may possess an ability to connect to a packet data network 175 that the wearable device 105 may not have. In this case, the user mobile device 145 may be capable of updating its own memory with information from the various networks 155. The user mobile device 145 may then be instructed to relay the information downloaded from the networks 155 to the wearable device 105 to update, for example, the side effects local database 143. In other embodiments, the user mobile device 145 may be instructed to perform the evaluation and the detection of one or more known side effects upon receiving sensor data from the wearable device 105.

The user mobile device 145 may also include an operating system (OS) 150. The OS 150 is software module that can be used to manage the various elements and resources associated with the user mobile device 145. Exemplary OS 150 that may be used with the user mobile device 145 include Darwin, RTXC, LINUX, UNIX, OS X, ANDROID, WINDOWS, or an embedded operating system such as VxWorks.

As noted above, the wearable device 105 and/or the user mobile device 145 may be capable of connecting to the packet data network 175 in order to access information from one or more networks 155. As illustrated in FIG. 1, there are many different networks that may be available. Exemplary networks may include a wearable device vendor network, a pharmacist/doctor network, and a drug company network.

Each of the networks may be managed and updated by a particular entity. For example, the wearable device vendor network, for example, can be a network set up by a particular vendor for the wearable device used by the user. Meanwhile, the pharmacist/doctor network may be set up and managed by a particular pharmacist/doctor network. The drug company network may be set up and managed by a particular drug company.

Each network may include information (e.g., known side effects, rules and algorithms for detecting the one or more known side effects, software module for particular devices) related to one or more different drugs available in the market that the user may use. For example, the drug company may include information related to the drugs that it manufactures and sells. In contrast, the pharmacist/doctor network may provide a broader range of information for the drugs that a particular pharmacist or doctor may be capable of prescribing to a patient. The wearable device vendor network may include different types of software module that can be downloaded and used within each of the devices (e.g., wearable, mobile).

Generally, each of the various networks 155 may include network side effects software module 195, a network side effect database 200, user application programming interface (API) and a 3^{rd} party API 160. As disclosed above, the information that each network has (e.g., software module, side effect database) may be provided by the entity that sets up and manages the particular network. In some embodiments, the information may also be provided by third party networks. For example, there may be third parties (e.g., health organizations) that may specialize in studying effects of a particular drug. In these cases, these third parties may be allowed to update the information in the various networks 155. To do so, the 3^{rd} party API may be used to facilitate communication between the third party network with the networks 155. It should be noted, in some embodiments, that some form of authorization may be necessary before the third party (via the third party network 170) can upload information to a network 155. This may be implemented to prevent false or misleading information from being loaded onto the networks and to ensure that the information that is provided is from a trustworthy source. The user, on the other hand, may utilize the user API 165 to transmit requests to a network for information and subsequently receive the requested information from the particular network 155.

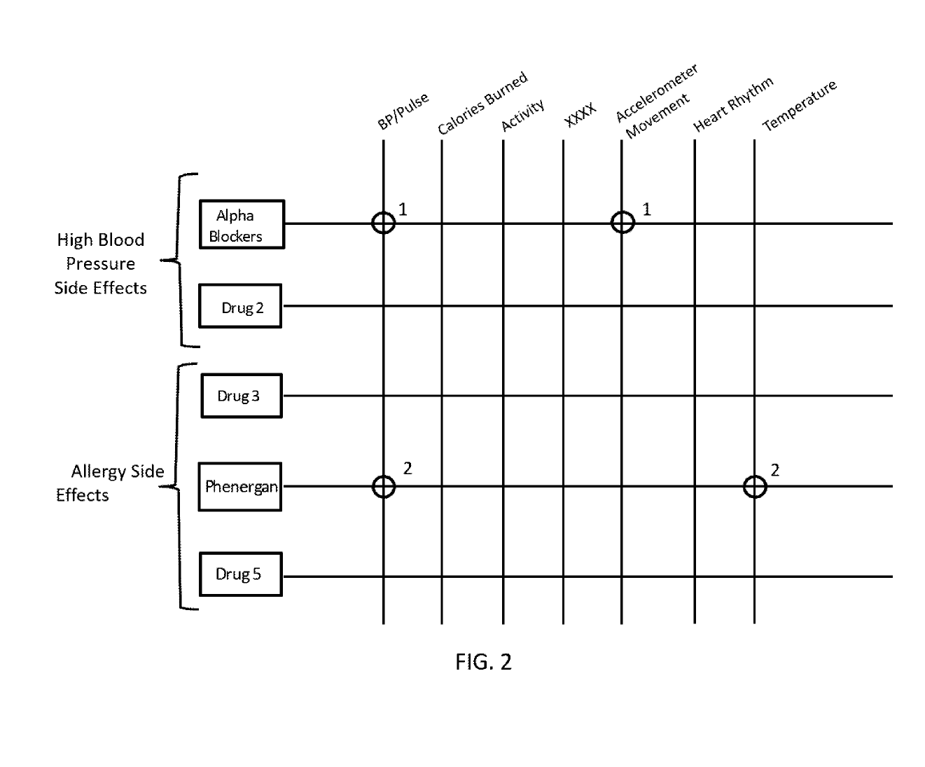

FIG. 2 shows a grid illustrating exemplary prescription drugs and their associated one or more known side effects that may be monitored by the present invention. As indicated above, various drugs (e.g., prescription drugs) that a user can use may have one or more different side-effects. Depending on a particular side-effect, one or more different vital signs measurement parameters (e.g., blood pressure, pulse, temperature) may be monitored.

As illustrated in the figure, some exemplary drugs are provided along the vertical axis of the grid: Alpha Blockers, Drug 2, Drug 3, Phenergan, and Drug 5. It may be possible to categorize the different drugs that are monitored into different groups/brackets based on the type of known side effect (e.g., high blood pressure or allergy). It should be noted that different types of drugs as well as different types of known side effects may also be included in the grid of FIG. 2.

With continued reference to the grid of FIG. 2, the horizontal axis may be used to identify a variety of different type of body metrics (e.g., vital signs measurement parameters) that the wearable device can monitor, measure, and record (e.g., blood pressure/pulse, calories burned, activity, temperature). One or more of these vital signs measurement parameters may be helpful in monitoring and detecting a known side effect. It should be noted that other types of vital signs measurement parameters may also be included in the grid. Furthermore, it should also be noted that different combinations of different drugs and vital signs measurement parameters can be included in this grid as necessary for the monitoring and detection of one or more known side effects for the various drugs that the user may use.

To further explain how one can interpret the information provided in the grid, discussion will be provided with respect to example 1. In an exemplary situation, suppose the user is taking alpha blockers for treatment of hypertension. It is known that alpha blockers are a high blood pressure type medication that may have side effects relating to a user's blood pressure and activity level. In another situation, suppose the user is taking Phenergan for treatment of motion sickness. It is known that Phenergan is a type of allergy medicine that may affect a user's blood pressure and body temperature.

The grid of FIG. 2 illustrates the above exemplary situations relating to a combination of vital signs measurement parameters that can be used to monitor the health condition of the user in connection with the use of alpha blockers and/or Phenergan. The monitoring of the particular combination of vital signs measurement parameters may obtain sensor data that can be evaluated to produce a conclusion that one or more of these particular vital signs measurement parameters are abnormal. The abnormality of these vital signs measurement parameters may be linked to the use of a particular drug. Such combination of vital signs measurement parameters may be incorporated, for example, by the wearable device to be evaluated in order to detect the one or more known side effects. In other embodiments, an administrator of a network may implement the combination of vital signs measurement parameters into software module that may be designed to detect the one or more known side effects for the particular drug.

With continued reference with the first example, if the blood pressure of the user was evaluated to be higher than normal, this may be indicative that the known side effect of high blood pressure from use of alpha blockers has been detected. If this conclusion is reached, notification (via the wearable device and/or the user mobile device) may be provided, for example, to the user to inform the user of the potentially detected one or more known side effects. In some embodiments, notification may include suggestions to stop using the medicine or to see a doctor. These suggestions may aim to prevent, mitigate or resolve the one or more known side effects.

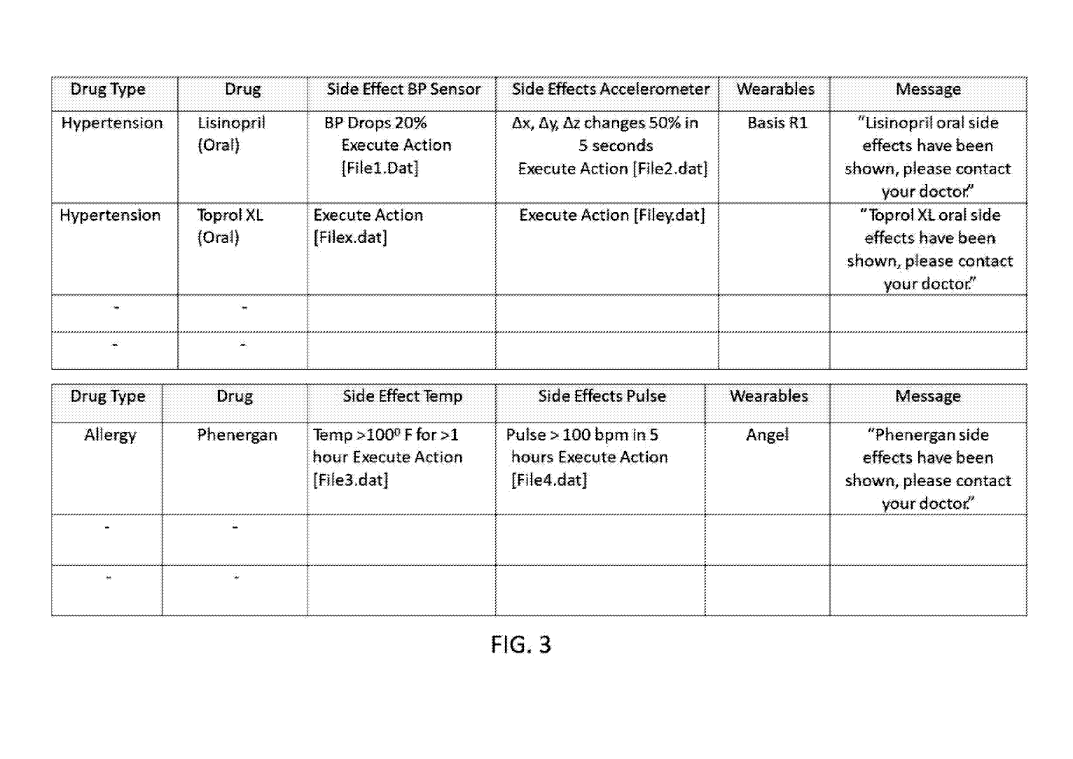

FIG. 3 illustrates an exemplary side effects database that may be found in the various networks. As indicated above, the side effects database may include information about a particular drug, the corresponding known side effects, what vital signs measurement parameters that may be monitored and rules and/or software module for detecting the one or more known side effects. In some embodiments, the side effects database may also include additional information such as how the prescription drug is taken, possible messages to be provided to the user if the known side effects are detected. It should be noted that additional types of information may also be stored in the side effects database.

With continued reference to the network side effects database, the user may be allowed to download all or selective portions of the database to be stored into the wearable device and/or user mobile device. For example, the user may request information (e.g., software module, known side effects, rules) regarding a particular drug that the user is taking. In other situations, the user may be able to request all available information stored within the network side effects database. In any case, the requested information may be provided by the various networks to the user to update the wearable device and/or user mobile device. The information can then be used by the user to monitor and detect one or more known side effects through use of the wearable device and user mobile device.

As illustrated in FIG. 3, a first exemplary side effects database may be provided for the prescription drugs directed at treating hypertension. In particular, the table includes an entry for Lisinopril. The entry also includes related information with respect to Lisinopril corresponding to how the drug is taken (e.g., orally) as well as rules/algorithms for detecting one or more known side effects (e.g., blood pressure) for taking the drug. For example, with respect to the blood pressure side effect, there is an exemplary rule that recites that a first action file (“File1.Dat”) is to be executed when a measured blood pressure/heart rate (e.g., from a blood pressure sensor) has dropped 20%. The first action file may correspond to a graphic, alert or message that may be provided to the user to inform them that the known side effect for high blood pressure has been detected. Also recited with Lisinopril is a second rule specifying execution of a second action file (“File2.dat”) when there has been a measured change in motion by the user (e.g., from an accelerometer) indicated by a change in the x, y and z axis greater than 50% within a five second time period. The accelerometer, in this situation, may be used to detect a scenario where the change in motion could be correlated with the user falling. In this way, the second action that is executed may again be related to a graphic, alert or message that is provided to the user to notify the possible cause of the fall. In some embodiments, the wearable device and/or user mobile device may be capable of contacting other parties (e.g., doctors, relatives, hospital, emergency services) based on the detection of the one or more known side effects using the rules stored in the side effects database.

The second exemplary side effects database of FIG. 3 provides information for detection of one or more known side effects regarding the allergy drug Phenergan. With this database, similar to the first exemplary side effects database, information regarding Phenergan includes the vital signs measurement parameters (e.g., user temperature and pulse) that are used to monitor/detect the one or more known side effects. For example, there are rules that that are directed at monitoring the body temperature and pulse of the user to detect one or more known side effects from using Phenergan. If the user has a body temperature over one hundred degrees Fahrenheit for over an hour or if their pulse rate is over one hundred beats per minute for over five hours, the side effects database may instruct the execution of corresponding files (File3.dat and File4.dat, respectively). As with the example in Lisinopril, notification can be provided to the user (via the wearable device and/or user mobile device) or to other parties (e.g., hospital) upon detection of the one or more known side effects from the use of Phenergan.

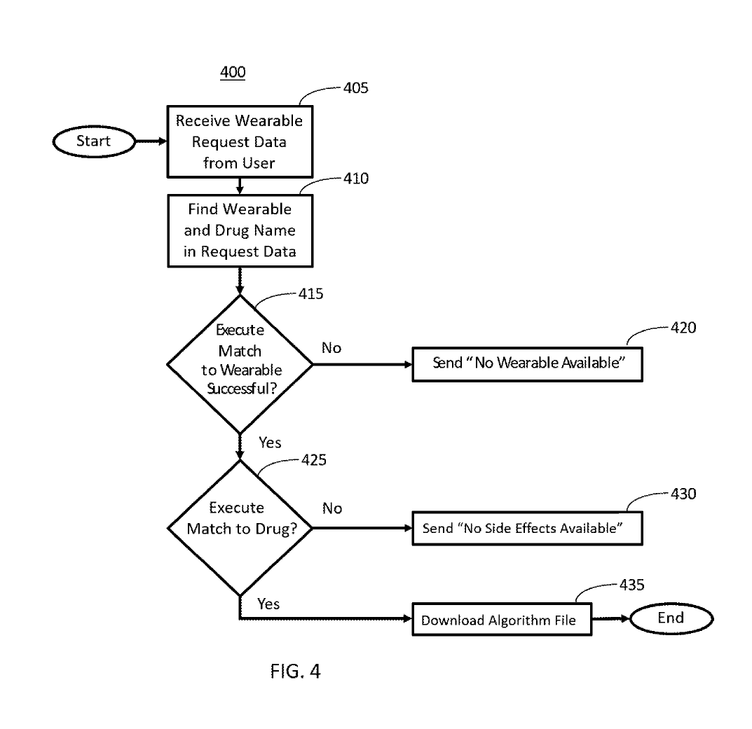

FIG. 4 shows an exemplary method 400 for the network side effects software module that can be found on the various networks. As indicated above, the network side effects software module can initially be stored in the various networks. In fact, there may be various different types of network side effects software module directed at monitoring different known side effects for different drugs.

With the exemplary method 400 illustrated in FIG. 4, a single user would be able to search for and/or request the appropriate information to be downloaded from the various networks to be stored on the wearable device and/or user mobile device. In particular, the method 400 for the side effects software module included in the various networks can facilitate providing the appropriate information to the user for use on their respective device.

In step 405, the network side effects software module may receive a request from the user. The request may be provided, for example, from the wearable device and/or the user mobile device. The request may also specify a particular drug that the user would like information about in order to monitor and detect the one or more known side effects associated with the drug. In one embodiment, the user may be capable of inputting the particular drug into the device (e.g., wearable device and/or user mobile device). The user input can then be relayed to the various networks to see if related information about that drug can be provided back to the user. In other embodiments, the user may request updating the wearable device and/or user mobile device with all available information stored in the particular network.

In step 410, the network side effects software module can then check the network side effects database in the network to determine if any information related to the drug being requested can be found. In some embodiments, the network side effects software module can also check to see if the user wearable device and/or user mobile device model is also found in the network side effects database. A particular model for the wearable device and/or user mobile device may be included in the network side effects database in order to ensure that software module that is only compatible with a particular OS for example is provided to the devices that can run the software module.

In step 415, an evaluation is first conducted to see if the particular network that is being contacted with the user request found a match with the user device (e.g., wearable device and/or user mobile device). If no match was found, a message may be provided from the network to the user indicating that “no wearable was available” as seen in step 420. This message may coincide with a situation where the user device (e.g., wearable device and/or user mobile device) is not compatible with the software module stored in the network. In another situation, the message may also coincide with the use of a wearable device and/or user mobile device that has not yet been configured to operate with the network or information stored in the network (e.g., side effects software module). If there is a match with the user device, however, the side effects software module can proceed to the next step.

In step 425, assuming that a user device (e.g., wearable device and/or user mobile device) was detected and recognized by a network, the network side effects software module can then search the network side effects database for related information regarding the drug that that the user requested. It may be possible, however, that a network that the user contacted does not have any information about a particular drug. If that is the case, corresponding to the network side effects software module not finding any information about the requested drug, the network side effects software module can provide a message to the user indicating that “No Side Effects Available” in step 430. If that is the case, the user may send the request to another network to see if the other network has any information related to the drug in question.

If there is a match, however, the network side effects software module may provide the requested information to the user (e.g., corresponding side effects software module, rules/algorithms in the side effects database) in step 435. The network side effects software module may provide the requested information through the user API and store the information in the memory of the device that sent the request (e.g., wearable device and/or the user mobile). Once the requested information has been provided to the user, the network side effects software module can terminate until at least another request is provided from another user.

FIG. 5 shows an exemplary method 500 for the side effects software module found on the user device. As described above, the user may use the wearable device and/or the user mobile device to evaluate sensor data to detect the onset of one or more known side effects from using one or more drugs. The method 500 illustrated in the figure shows the steps that the user device (e.g., wearable device and/or user mobile device) performs to monitor and detect the one or more known side effects.

In step 505, the user loads information into the user device (e.g., wearable device and/or user mobile device). In particular, the user may load information regarding the drug or drugs the user plans on taking that the user desires to monitor and detect for one or more known side effects. Information about the devices (e.g., wearable device and/or user mobile device) may also be included. For example, if the user is inputting information about the drugs that the user plans on using into the wearable device, the user may also want to include information about any associated user mobile devices that the user may also wish to use in conjunction. The information inputted into the user device (e.g., wearable device and/or user mobile device) may be helpful in determining whether a particular network can provide any information to the user device related to the drug. The information may also be used in determining if the information (e.g., side effects software module, rules, algorithms) stored in the network is compatible with a particular user device.

In step 510, the user provides the information regarding the drug and/or user devices to one or more networks found in the packet data network. As described above in FIG. 4, the network side effect software module of the network uses the information provided by the user in order to determine if any related information may be transmitted back to the user. For example, the networks may check their respective side effects database for any information that the user might find useful based on the information requested provided by the user.

In step 515, the user receives the requested information from one or more networks that were contacted in step 510. The requested information may include, for example, information about the drug (e.g., known side effects, instructions for use). The requested information may also include rules and/or algorithms that can be used by the user device (e.g., wearable device and/or user mobile device) to monitor the user health condition and detect when the one or more known side effects arise. The requested information can then be stored in the side effects local database of the user device.

In step 520, the base software module of the user device (e.g., wearable device and/or the user mobile device) is run. The base software module may begin polling the sensors associated with the wearable device to obtain sensor data. In particular, based on the information received from the various networks in step 515, the base software module may be instructed to poll for particular sensor data pertaining, for example, to poll for vital signs measurement parameters that may be used to detect a particular known side effect. The relevant sensor data can then be obtained by the wearable device and stored in memory for later use.

In step 525, the side effects software module can then determine if there are any existing rules and actions that applicable. In particular, the side effects software module can evaluate the sensor data obtained in step 520 along with any information that is stored in the side effects local database of the user device. The information stored in the side effects local database may be the same information received from the various networks in step 515.

If there are no existing rules and actions applicable, this may coincide with a situation that the health of the user is normal (e.g., no known side effects have been detected based on the sensor data that was evaluated). The side effects software module can then loop back to step 520 to have the base software module continue running. This may instruct the wearable device to continue polling for sensor data that can be used to provide a further updated evaluation of the user health condition. It should be noted that the loop can be performed at regular intervals or may be continually occurring. In any case, the loop allows the user to obtain up-to-date monitoring of their health condition.

In a situation where at least one rule and action is applicable, the side effects software module may proceed to step 530. The situation where at least one rule and action is applicable may coincide with the wearable device obtaining sensor data on one or more vital signs measurement parameters of the user that have been evaluated as being indicative of one or more known side effects. When one or more known side effects have been detected, the user device can execute the action, for example, that was stored in the side effect database. The action may include notifying the user of the detected one or more known side effects. The notification may also include a message for the user. The message may include, for example, information about the user health condition, information about the detected one or more known side effects, and any actions that the user may perform (e.g., call the doctor, stop taking the drug) in order to mitigate or resolve the effects of the detected one or more known side effect.

According to one embodiment, the notification to the user can include a predetermined adjustment or alteration to a drug delivery regime based on the detected one or more side effects. The predetermined adjustment or alteration to the drug delivery regime may be a user controlled or a connected pill dispensing device being adjusted or altered to dispense a different quantity or frequency of drug. The notification may further request the user to confirm that the drug delivery regime has been adjusted or altered according to the predetermined adjustment or alteration.

The advantage of using predetermined adjustment or alteration to a drug delivery regime based on the detected one or more side effects is that the adjustment or alteration to the drug delivery regime can help eliminate or reduce the severity of the detected one or more side effects before the side effects get worse. For example, if medical personnel are not available to monitor a patient continuously, for example, at night, the predetermined adjustment of a drug delivery in response to a detected side effect or symptom can help ensure that the unattended patient's condition will not worsen until such time when a medical personnel becomes available to check on the patient's condition.

Once the user has been informed of the detected side effect, the side effects software module can then loop back to the base software module in step 520 in order to obtain additional sensor data. Similar to the loop back performed in step 525, the base software module may be instructed to continue polling for sensor data that can be used to provide a further updated evaluation of the user health condition. The loop back to the base software module for additional sensor data can also be used to evaluate whether a condition associated with the detected side effect continues to persist, has improved or worsened. The user may be also informed of the continued existence of the detected one or more known side effects and may be instructed to perform various actions (e.g., call the doctor, call 911) based on the severity of the detected side effect.

The side effects software module may continue to run so long as the wearable device is in use to obtain sensor data used to monitor and detect one or more known side effects of drugs the user is using. In some embodiments, the wearable device may continually provide sensor data to the user mobile device. The user mobile device can then evaluate the sensor data against the information about the drug stored in the side effects network database. In this way, steps 520-530 can be performed between both the wearable device and the user mobile device.

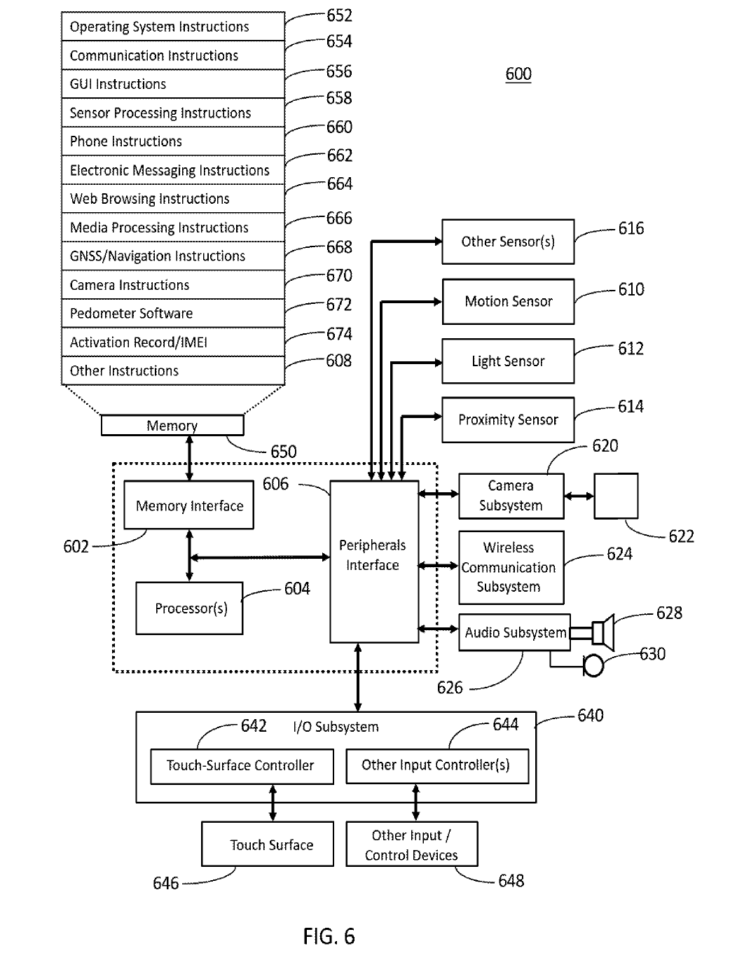

FIG. 6 illustrates an exemplary computing device 600 that may be utilized to implement the various features and processes described herein. For example, the computing device 600 could be implemented in a pedometer. Device 600 as illustrated in FIG. 6 includes memory interface 602, processors 604, and peripheral interface 606. Memory interface 602, processors 604 and peripherals interface 606 can be separate components or can be integrated as a part of one or more integrated circuits. The various components can be coupled by one or more communication buses or signal lines.

Processors 604 as illustrated in FIG. 6 is meant to be inclusive of data processors, image processors, central processing unit, or any variety of multi-core processing devices. Any variety of sensors, external devices, and external subsystems can be coupled to peripherals interface 606 to facilitate any number of functionalities within the architecture of the exemplar mobile device 600. For example, motion sensor 610, light sensor 612, and proximity sensor 614 can be coupled to peripherals interface 606 to facilitate orientation, lighting, and proximity functions of the mobile device. For example, light sensor 612 could be utilized to facilitate adjusting the brightness of touch surface 646. Motion sensor 610, which could be exemplified in the context of an accelerometer or gyroscope, could be utilized to detect movement and orientation of the mobile device. Display objects or media could then be presented according to a detected orientation (e.g., portrait or landscape).

Other sensors 616 could be coupled to peripherals interface 606, such as a temperature sensor, a vital signs measurement sensor, or other sensing device to facilitate corresponding functionalities. Location processor (e.g., a global positioning transceiver) can be coupled to peripherals interface 606 to allow for generation of geo-location data thereby facilitating geo-positioning. An electronic magnetometer such as an integrated circuit chip could in turn be connected to peripherals interface 606 to provide data related to the direction of true magnetic North whereby the mobile device could enjoy compass or directional functionality. Camera subsystem 620 and an optical sensor 622 such as a charged coupled device (CCD) or a complementary metal-oxide semiconductor (CMOS) optical sensor can facilitate camera functions such as recording photographs and video clips.

Communication functionality can be facilitated through one or more communication subsystems 624, which may include one or more wireless communication subsystems. Wireless communication subsystems 624 can include 802.x or Bluetooth transceivers as well as optical transceivers such as infrared. Wired communication system can include a port device such as a Universal Serial Bus (USB) port or some other wired port connection that can be used to establish a wired coupling to other computing devices such as network access devices, personal computers, printers, displays, or other processing devices capable of receiving or transmitting data. The specific design and implementation of communication subsystem 624 may depend on the communication network or medium over which the device is intended to operate. For example, a device may include wireless communication subsystem designed to operate over a global system for mobile communications (GSM) network, a GPRS network, an enhanced data GSM environment (EDGE) network, 802.x communication networks, code division multiple access (CDMA) networks, or Bluetooth networks. Communication subsystem 624 may include hosting protocols such that the device may be configured as a base station for other wireless devices. Communication subsystems can also allow the device to synchronize with a host device using one or more protocols such as TCP/IP, HTTP, or UDP.

Audio subsystem 626 can be coupled to a speaker 628 and one or more microphones 630 to facilitate voice-enabled functions. These functions might include voice recognition, voice replication, or digital recording. Audio subsystem 626 in conjunction may also encompass traditional telephony functions.

I/O subsystem 640 may include touch controller 642 and/or other input controller(s) 644. Touch controller 642 can be coupled to a touch surface 646. Touch surface 646 and touch controller 642 may detect contact and movement or break thereof using any of a number of touch sensitivity technologies, including but not limited to capacitive, resistive, infrared, or surface acoustic wave technologies. Other proximity sensor arrays or elements for determining one or more points of contact with touch surface 646 may likewise be utilized. In one implementation, touch surface 646 can display virtual or soft buttons and a virtual keyboard, which can be used as an input/output device by the user.

Other input controllers 644 can be coupled to other input/control devices 648 such as one or more buttons, rocker switches, thumb-wheels, infrared ports, USB ports, and/or a pointer device such as a stylus. The one or more buttons (not shown) can include an up/down button for volume control of speaker 628 and/or microphone 630. In some implementations, device 600 can include the functionality of an audio and/or video playback or recording device and may include a pin connector for tethering to other devices.

Memory interface 602 can be coupled to memory 650. Memory 650 can include high-speed random access memory or non-volatile memory such as magnetic disk storage devices, optical storage devices, or flash memory. Memory 650 can store operating system 652, such as Darwin, RTXC, LINUX, UNIX, OS X, ANDROID, WINDOWS, or an embedded operating system such as VxWorks. Operating system 652 may include instructions for handling basic system services and for performing hardware dependent tasks. In some implementations, operating system 652 can include a kernel.

Memory 650 may also store communication instructions 654 to facilitate communicating with other mobile computing devices or servers. Communication instructions 654 can also be used to select an operational mode or communication medium for use by the device based on a geographic location, which could be obtained by the GNNS/Navigation instructions 668. Memory 650 may include graphical user interface instructions 656 to facilitate graphic user interface processing such as the generation of an interface; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GNNS/Navigation instructions 668 to facilitate GPS and navigation-related processes, camera instructions 670 to facilitate camera-related processes and functions; pedometer software 672, activation record/IMEI 674, and instructions 608 for any other application that may be operating on or in conjunction with the mobile computing device. Memory 650 may also store other software instructions for facilitating other processes, features and applications, such as applications related to navigation, social networking, location-based services or map displays.

Each of the above identified instructions and applications can correspond to a set of instructions for performing one or more functions described above. These instructions need not be implemented as separate software programs, procedures, or modules. Memory 650 can include additional or fewer instructions. Furthermore, various functions of the mobile device may be implemented in hardware and/or in software, including in one or more signal processing and/or application specific integrated circuits.

Certain features may be implemented in a computer system that includes a back-end component, such as a data server, that includes a middleware component, such as an application server or an Internet server, or that includes a front-end component, such as a client computer having a graphical user interface or an Internet browser, or any combination of the foregoing. The components of the system can be connected by any form or medium of digital data communication such as a communication network. Some examples of communication networks include LAN, WAN and the computers and networks forming the Internet. The computer system can include clients and servers. A client and server are generally remote from each other and typically interact through a network. The relationship of client and server arises by virtue of computer programs running on the respective computers and having a client-server relationship to each other.

One or more features or steps of the disclosed embodiments may be implemented using an API that can define on or more parameters that are passed between a calling application and other software code such as an operating system, library routine, function that provides a service, that provides data, or that performs an operation or a computation. The API can be implemented as one or more calls in program code that send or receive one or more parameters through a parameter list or other structure based on a call convention defined in an API specification document. A parameter can be a constant, a key, a data structure, an object, an object class, a variable, a data type, a pointer, an array, a list, or another call. API calls and parameters can be implemented in any programming language. The programming language can define the vocabulary and calling convention that a programmer may employ to access functions supporting the API. In some implementations, an API call can report to an application the capabilities of a device running the application, such as input capability, output capability, processing capability, power capability, and communications capability.

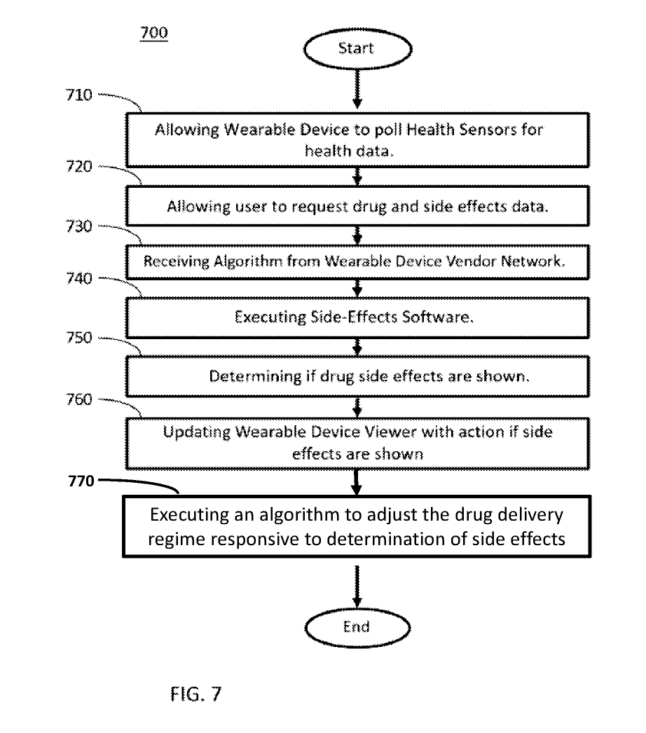

FIG. 7 illustrates an exemplary method 700 for monitoring and detecting one or more known side effects of drugs. The method 700 of the present invention, as discussed above, may be carried out through the use of the wearable device and the user mobile device. In some embodiments, the method 700 of the present invention may be performed solely on the wearable device.

In step 710, the wearable device polls for sensor data from the various sensors associated with the wearable device. The sensor data may be related to one or more vital signs measurement parameters (e.g., pulse, blood pressure, temperature) that can be used to evaluate on a user device (e.g., wearable device and/or user mobile device) whether one or more known side effects has been detected.

In step 720, the user may request information from the various networks regarding one or more drugs that the user is currently taking. By using the wearable device and/or mobile device, the user can provide the various networks with information about the drug and the devices that may be used to monitor and detect the one or more known side effects. The various networks can check their side effects database to determine if there is any information about one or more known side effects (e.g., rules and algorithms for detection). A check may also be performed to determine if the user device is compatible with the requested information.

In step 730, the requested information from the various networks can be provided to the user. The information can be provided, for example, to the device that the user provided the information request from (e.g., the wearable device or the user mobile device). The information can then be stored in the side effects database for that particular device. The information that may be received, as noted above, may include information about the identified drug, the one or more known side effects for that drug and any ways (e.g., rules and algorithms) for detecting the one or more known side effects for the drug.

In step 740, the user device (e.g., wearable device or user mobile device) executes the side effect software module. In particular, the user device can evaluate the sensor data obtained with the information received from the various networks pertaining to a particular drug's one or more known side effects.

In step 750, based on the evaluation, the side effects software module can determine if the one or more known side effects has been detected. As discussed above, the information received from the various networks and stored in the side effects database may include various rules and algorithms that can be used to monitor one or more vital signs measurement parameters of the user. In a situation where one or more of the monitored vital signs measurement parameters is abnormal, the side effects software module may notify the user of the associated sensor data. The notification is beneficial because the abnormal sensor data may be indicative of a one or more known side effects being detected.

In step 760, the user device (e.g., wearable device or user mobile device) may notify the user of the detected known side effect. The notification may be based on the action associated with the detected one or more known side effects stored in the side effects database. The notification may include, for example, a message relating to the detected known side effect. The message may also provide instructions to the user on what should be done to mitigate or resolve the effects of the detected side effect (e.g., call the doctor, stop taking the drug). The notification may be provided on the display of the user device for the user to view.

In step 770, the user device (e.g., wearable device or user mobile device) may execute an algorithm to adjust the drug delivery regime responsive to the determination that one or more known side effects have been detected. The algorithm that may be executed may include, for example, a stored computer program for adjusting a drug delivery regime according to a predetermined adjustment or alteration in order to eliminate or reduce the severity of the detected one or more one or more side effects. According to one embodiment, the adjustment or alteration may be a reduction in quantity or frequency of drug delivery instances in the drug delivery regime. According to another embodiment, the adjustment or alteration may be an addition of a side-effect suppression drug into the drug delivery regime. According to another embodiment, the adjustment or alteration may be a change in the times of drug delivery instances in the drug delivery regime. According to a further embodiment, the adjustment or alteration may be a combination of the foregoing adjustments or alterations.

According to one embodiment, the notification to the user can include a predetermined adjustment or alteration to a drug delivery regime based on the detected one or more side effects. The predetermined adjustment or alteration to the drug delivery regime may be a user controlled or a connected pill dispensing device being adjusted or altered to dispense a different quantity or frequency of drug. The notification may further request the user to confirm that the drug delivery regime has been adjusted or altered according to the predetermined adjustment or alteration.

In other embodiments, the notification may be provided from the user device to other entities (e.g., hospitals, emergency, relatives, caregivers) upon detection of the known side effect.

Upon obtaining the requested information about the prescription drug, the network server may provide the information to the wearable device. Such information may include data on side effects and algorithms used for determining if side effects are present. In particular, side effects may be determined through execution of the side effects software module found in the user device, thereby using body metrics measured by the plurality of sensors on the wearable device and running the measured data through the algorithms associated with the prescription drug. Based on the output from the algorithm, a message can be provided to the user on the viewer (e.g., display) of the user device, as well as instructions as to what needs to be done (e.g., to contact a doctor for assistance).

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen in order to best explain the principles of the technology and its practical application to thereby enable others skilled in the art to best utilize the technology in various embodiments and with various modifications as are suited to the particular use contemplated. It is intended that the scope of the technology be defined by the claim.

1. A computer-implemented method for monitoring and detecting one or more known side effects of drugs using a wearable device, the method comprising:
polling for sensor data from a wearable device; the sensor data indicating one or more vital signs measurement parameters of a user, wherein the sensor data is used for evaluating whether a one or more known side effects has been detected;
transmitting a request for information to various networks, the information request including identification of at least one drug the user is using;
receiving the requested information from the various networks for monitoring and detecting one or more known side effects of the identified drug;
storing the received information from the various networks into memory of the wearable device;
evaluating, for each identified drug, the obtained sensor data with the stored information received from the various networks pertaining to that identified drug;
determining whether one or more known side effects have been detected, wherein the determination is based on the evaluation of the sensor data with the stored information about the identified drug;
executing one or more actions to notify the user and other third parties based on the detected one or more known side effects; and
executing an algorithm to adjust the drug delivery regime responsive to the determination that one or more known side effects have been detected.

2. The method of claim 1, wherein the vital signs measurement parameters include at least one of blood pressure, pulse, movement, and temperature.

3. The method of claim 1, wherein the information request also includes information about the wearable device that the user is using to monitor and detect one or more known side effects, the information about the wearable device being used to evaluate compatibility between the wearable device and the requested information being provided by the various networks.

4. The method of claim 1, wherein the requested information received from the various networks includes identification of at least one vital signs measurement parameter to be monitored whereby associated sensor data can be used to detect one or more known side effects of the drug.

5. The method of claim 1, wherein the requested information received from the various networks includes rules and algorithms directed at evaluating sensor data obtained and determining if the one or more known side effects has been detected based on the sensor data.

6. The method of claim 1, wherein the one or more actions to notify the user based on the detected one or more known side effects includes execution of an algorithm to transmit messages or perform actions that have been obtained from the various networks and subsequently stored in memory.

7. The method of claim 1, wherein other third parties may include doctors, hospitals, emergency services and relatives.

8. The method of claim 1, further comprising:
storing the received information from the various networks into memory of a user mobile device; and
transmitting the obtained sensor data to the user mobile device, wherein the user mobile device is also able to perform the evaluation of the obtained sensor data.

9. A wearable device for monitoring and detecting one or more known side effects of drugs, the wearable device comprising:
a plurality of sensors;
memory; and
a processor configured for:
polling for sensor data; the sensor data monitoring one or more vital signs measurement parameters of a user, wherein the sensor data is used for evaluating whether one or more known side effects has been detected;
transmitting a request for information to various networks, the information request including identification of at least one drug the user is using;
receiving the requested information from the various networks for monitoring and detecting one or more known side effects of the identified drug;
storing the received information from the various networks into memory;
evaluating, for each identified drug, the obtained sensor data with the stored information received from the various networks pertaining to that identified drug;
determining whether one or more known side effects have been detected, wherein the determination is based on the evaluation of the sensor data with the stored information about the identified drug;
executing one or more actions to notify the user and other third parties based on the detected one or more known side effects; and
executing an algorithm to adjust the drug delivery regime responsive to the determination that one or more known side effects have been detected

10. The wearable device of claim 9, wherein the information request also includes information about the wearable device that the user is using to monitor and detect one or more known side effects, the information about the wearable device being used to evaluate compatibility between the wearable device and the requested information being provided by the various networks.

11. The wearable device of claim 9, wherein the requested information received from the various networks includes identification of at least one vital signs measurement parameter to be monitored whereby associated sensor data can be used to detect one or more known side effects of the drug.

12. The wearable device of claim 9, wherein the one or more actions to notify the user based on the detected one or more known side effects includes transmitting messages or executing actions that have been obtained from the various networks and subsequently stored in memory.

13. The wearable device of claim 9, wherein the processor is further configured for:
storing the received information from the various networks into memory of a user mobile device; and
transmitting the obtained sensor data to the user mobile device; wherein the user mobile device is also able to perform the evaluation of the obtained sensor data.
